(19) United States
(12) Patent Application Publication
(10) Pub. No.: US 2003/0125300 A1
(43) Pub. Date: Jul. 3, 2003

(54) Method and composition for conjugating molecules to nucleic acids

(75) Inventor: David H. Lee, Medford, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 10/272,498

(22) Filed: Oct. 15, 2002

Related U.S. Application Data
(60) Provisional application No. 60/329,363, filed on Oct. 15, 2001.

(51) Int. Cl. A61K 31/7076; C07H 19/04

(52) U.S. Cl. 514/48; 536/26.1

(57) ABSTRACT

Thiolester-activated amino acids; thiolester-activated nucleotides; polypeptide-oligonucleotide fusions comprising a polypeptide of interest and a thiolester-activated nucleotide; methods of producing the thiolester-activated nucleotides of the present invention; methods of producing polypeptide-oligonucleotide fusions of the present invention; and methods in which the polypeptide-oligonucleotide fusions are used, such as methods of delivering nucleic acid molecules into cells and cell lines.

### RELATED APPLICATION(S)

This application claims the benefit of U.S. Provisional Application No. 60/329,363, filed Oct. 15, 2001 and entitled “Method and Composition for Conjugating Molecules to Nucleic Acids,” by David Lee. The entire teachings of the above application(s) are incorporated herein by reference.

### BACKGROUND OF THE INVENTION

Complexes or fusions of proteins and nucleic acids have a variety of uses, such as tools for studying molecular mechanisms and as means for delivering molecules into cells. A variety of methods have been described for producing peptide-oligonucleotide conjugates (Forget, D. et al., Chem. Eur. J., 2001, 7(18):3976-3984 (2001)). Each of these methods has limitations and an additional method would be useful.

### SUMMARY OF THE INVENTION

Described herein are thiolester-activated amino acids; thiolester-activated nucleotides; polypeptide-oligonucleotide fusions comprising a polypeptide of interest and a thiolester-activated nucleotide; methods of producing the thiolester-activated nucleotides of the present invention; methods of producing polypeptide-oligonucleotide fusions of the present invention; and methods in which the polypeptide-oligonucleotide fusions are used, such as methods of delivering nucleic acid molecules into cells and cell lines.

Thiolester-activated nucleotides of the present invention are represented by the formula:

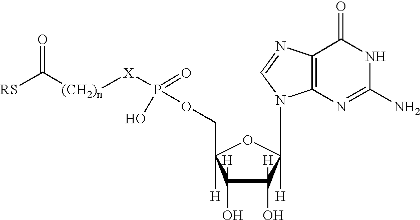

, wherein R is any hydrocarbon-containing group (straight or branched chain, cyclic) and X is a nucleophile, such as oxygen, nitrogen or sulfur. The nucleotide can be any nucleotide and in the formula presented above, the nucleotide represented is guanosine 5′ monophosphate (GMP). Thiolester-activated nucleotides of the present invention are produced, for example, as follows: An amino acid, which can be, for example, an aliphatic amino acid, such as glycine or alanine, or an aromatic amino acid, is activated as a thiolester and chemically conjugated to a nucleotide through the amino group of the amino acid, forming a phosphoamide bond. The resulting molecule, referred to as an activated-nucleotide fusion, is incorporated/linked (e.g., enzymatically, through in vitro transcription, such as through the action of an RNA polymerase, such as T7 RNA polymerase) to the 5′ end of a nucleic acid (e.g., RNA), such as to the 5′ end of guanosine 5′ monophosphate, thus producing a thiolester-activated oligonucleotide. The thiolester-activated oligonucleotide is coupled to any molecule, such as a polypeptide, that bears a cysteine residue in which the side chain and α-amine group are free and accessible for interaction with (reaction with) the nucleophile, such as a sulfur moiety. The resulting product is a fusion of the two components and, in the case in which the components are a polypeptide and a thiolester-activated nucleotide, the resulting product is a polypeptide-oligonucleotide fusion. Polypeptide-oligonucleotide fusions of the present invention are represented by the following formula:

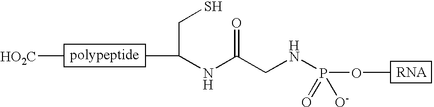

, wherein the polypeptide can be of any size (two or more amino acid residues) and the remainder of the fusion is the thiolester-activated oligonucleotide. The thiolester-activated oligonucleotide can be of any size (one or more nucleic acid residues). The polypeptide can be any of a variety of types, such as a polypeptide that facilitates delivery into cells and, thus, can be used to facilitate delivery of the nucleotide component of the fusion into cells. For example, the polypeptide component can be HIV tat protein, NF-κB/IκB, antennapedia protein or other proteins with positive charges, which facilitates transfer of nucleotides into cells or into the cell nucleus. Such fusions can be used, for example, for gene therapy, in which DNA or RNA encoding a polypeptide of interest is introduced into cells. Alternatively, the fusions can be used to introduce antisense RNA or DNA into cells, in which they act by hybridizing with a target (e.g., regulatory segment, encoding segment of a target gene) and interfere with its function. The fusions can also be used for RNA interference (RNAi) by making RNA enzymatically as a precursor, attaching to it a polypepetide (e.g., HIV tat protein) that facilitates its entry into cells and introducing the fusion into cells, in which it is processed.

### BRIEF DESCRIPTION OF THE DRAWINGS

FIG. 1A is a schematic representation of synthesis of a thiolester-activated nucleotide of the present invention.

FIG. 1B is a schematic representation of incorporation of thiolester conjugated guanosine monophosphate into an RNA pool.

FIG. 2 is a schematic representation of one embodiment of a thiolester-activated nucleotide, specifically thiolester-activated guanosine 5′ monophosphate.

FIG. 3 is a schematic representation of production of a polypeptide-RNA fusion of the present invention.

### DETAILED DESCRIPTION OF THE INVENTION

The present invention relates to thiolester-activated amino acids; thiolester-activated nucleotides; polypeptide-oligonucleotide fusions comprising a polypeptide of interest and a thiolester-activated nucleotide; methods of producing the thiolester-activated nucleotides of the present invention; methods of producing polypeptide-oligonucleotide fusions of the present invention; and methods in which the polypeptide-oligonucleotide fusions are used, such as methods of delivering nucleic acid molecules into cells and cell lines.

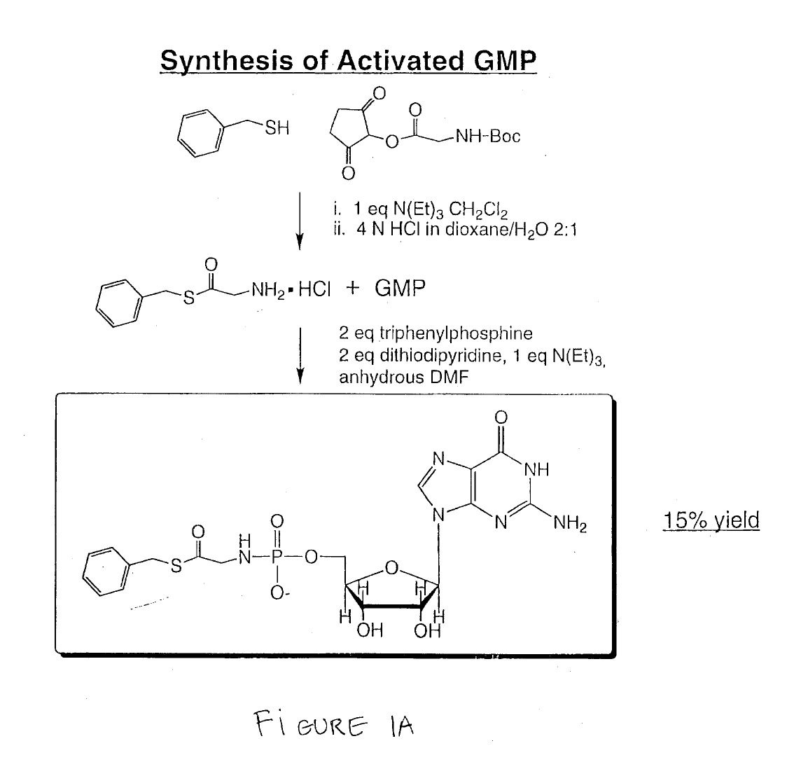

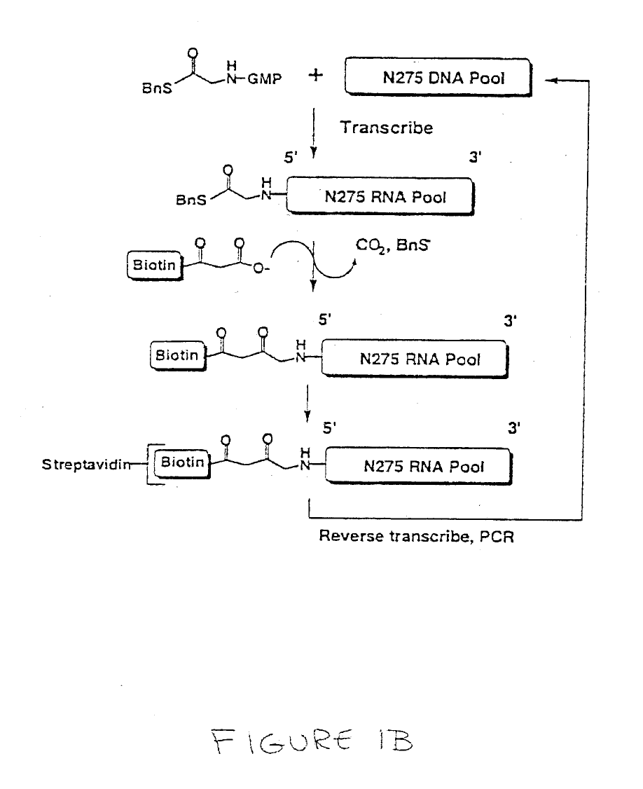

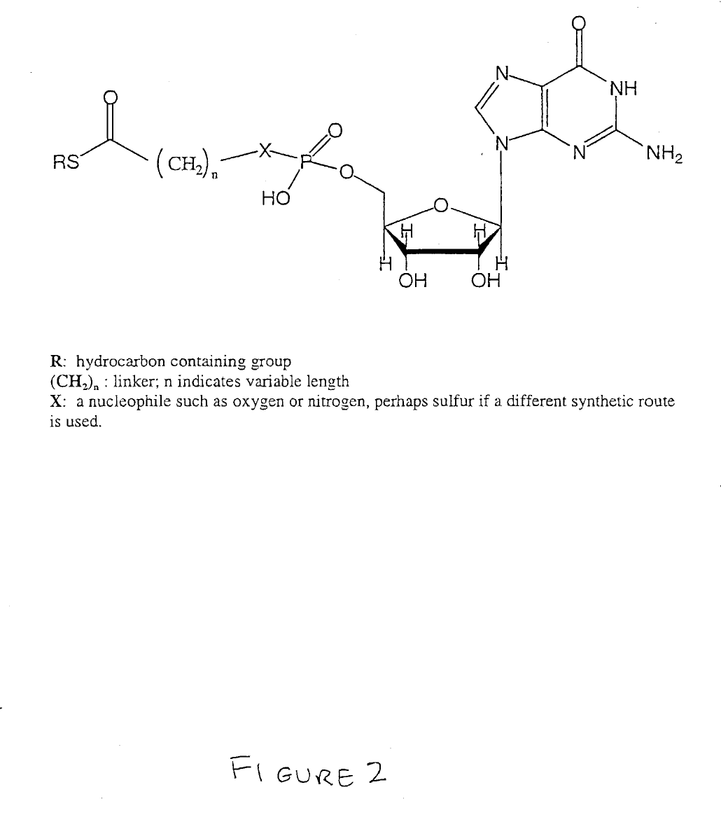

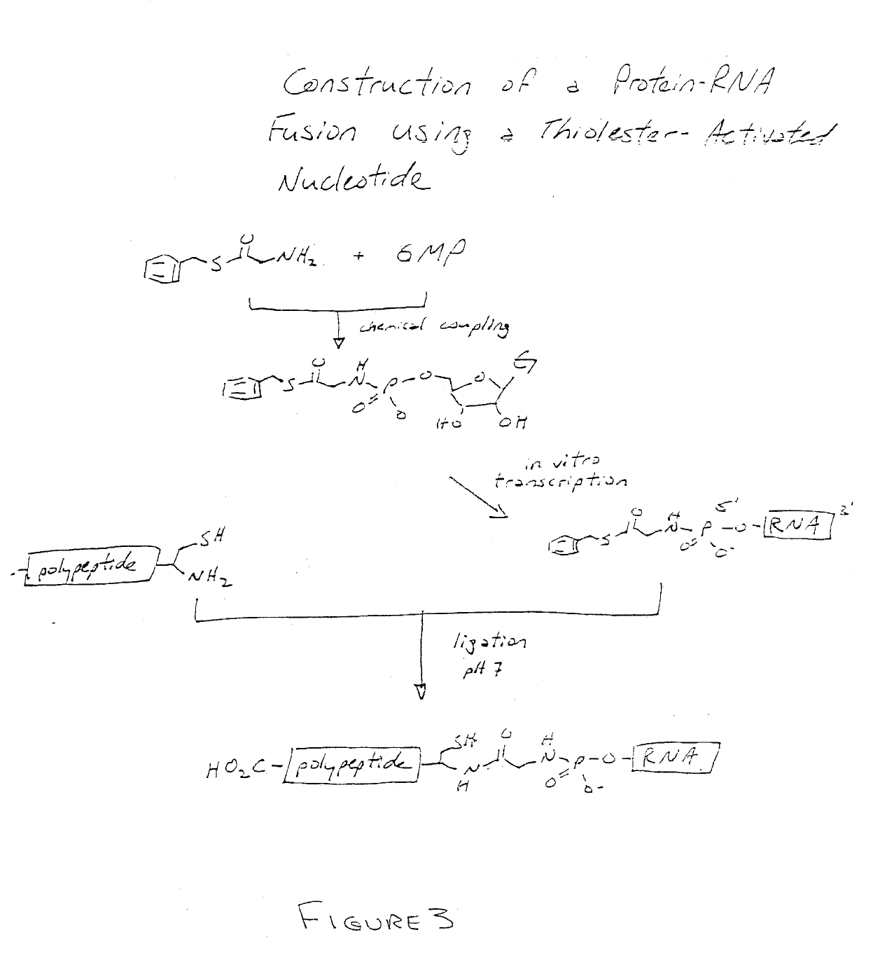

Synthesis of thiolester-activated nucleotides (such as thiolester-activated guanosine 5′ monophosphate) can be carried out by activating an amino acid, such as glycine or alanine, as a thiolester and chemically conjugating the resulting activated amino acid to a nucleotide (such as guanosine 5′ monophosphate or other nucleotide) through its amino group. In one embodiment, thiolester-activated nucleotides are produced as represented schematically in FIGS. 1A-1B. One embodiment of a thiolester-activated nucleotide of the present invention is represented schematically in FIG. 2. The resulting activated nucleotides are combined with reagents necessary for in vitro transcription (e.g., an appropriate RNA polymerase, such as T7 RNA polymerase and the four nucleotides that are present in RNA) under conditions (e.g., time, temperature, buffer conditions) under which in vitro transcription occurs, resulting in production of thiolester-activated oligonucleotides (thiolester-activated RNA). This product can, in turn, be coupled (e.g., under mild aqueous conditions) to a molecule such as a polypeptide. This process is represented schematically in FIG. 3. The resulting product is a polypeptide-oligonucleotide fusion of the present invention. The polypeptide component comprises at least two (two or more) amino acid residues. The amino acid residues can be L- or D-amino acid residues and can be naturally occurring or non-naturally occurring (modified). They can be obtained from sources in which they occur in nature or synthesized, using known methods, such as recombinant DNA technology or chemical synthetic methods. The oligonucleotide component is typically RNA, but can also be a RNA-DNA hybrid. It can be of any length and comprises at least two (two or more) nucleotides.

Polypeptide-oligonucleotide fusions of the present invention can be used, for example, to deliver oligonucleotides into cells. They are useful, for example, for delivering oligonucleotides that have therapeutic, prophylactic or diagnostic effects in the cells.

Exemplification

Synthesis of a thiolester conjugated guanosine monophosphate and its incorporation into an RNA pool for selection of a Ribozyme that catalyzed the Claisen condensation

Incorporation of the thiolester functionality into an RNA pool can be done enzymatically but requires the synthesis of a modified nucleotide. Such a nucleotide has been produced by condensing glycine thiobenzylester with guanosine 5′-monophosphate via Mukaiyama's & Hashimoto's oxidation-reduction condensation (Mukaiyama, T. & Hashimoto, M., Bull. Chem. Soc. Japan, 44:196-199 (1971)) to give the desired phophoamide conjugate in 15% yield (see FIG. 1A). The biotinylated β-ketoacid substrate is also needed. It was synthesized by simply reacting ethyl-4-chloroacetoacetate with a biotin derivative that contains a free thiol. Subsequent removal of the ester was achieved by using pig liver esterase.

The strategy for selecting the desired ribozymes is depicted in FIG. 1B. The thiolester conjugated guanosine monophosphate is incorporated into the 5′ end of a random RNA pool via T7 RNA polymerase transcription. To determine if the modified nucleotide was incorporated, the RNA is digested with the 5′ exonuclease xrn-l, which requires a single 5′ phosphate in order for it to digest RNA. If the thiolester group is at the end, the RNA should be protected. This pool will be incubated in the presence of a biotinylated β-keto acid. Those sequences that can decarboxylate the substrate and become self modified with biotin are selected by binding to immobilized streptavidin. Selected RNAs are reversed transcribed, amplified by PCR, and then transcribed again for another round of selection.

Selection of RNA sequences by this method simply means that the RNAs carry a biotin group. To verify that the biotin resides at the 5′ end and not internally, one can treat the RNA with glacial acetic acid, which cleaves the phophoamide bond, thereby liberating the correctly attached biotin. This RNA, which was originally selected by strepavidin binding will no longer bind strepavidin. Furthermore, full digestion of the oligonucleotide and TLC analysis of the nucleotides should yield 5 spots, 4 corresponding to canonical nucleotides and the fifth one being the biotinylated GMP. Its identity is verified by comparison to a standard. The catalytic efficiency of representative members of selected RNAs will be evaluated.

Once it is determined that the ribozymes have the correct activity, they (particularly those exhibiting the best activity) are reentered into a doped selection to 1) improve their catalytic ability and 2) identify the critical residues for activity through covariation of the sequence. Their catalytic efficiencies are measured, they are also tested to see if they can catalyze the Claisen condensation in trans and with turnover. Furthermore, the possibility of a processive ribozyme, which adds successive 2 carbon units to a growing chain, will be explored.

Bartel, D. P. and Unrau, P. J., Trends Cell Biol., 9:M9-M13 (1999).

While this invention has been particularly shown and described with references to preferred embodiments thereof, it will be understood by those skilled in the art that various changes in form and details may be made therein without departing from the scope of the invention encompassed by the appended claims.

What is claimed is:

1. A thiolester-activated nucleotide of the formula: 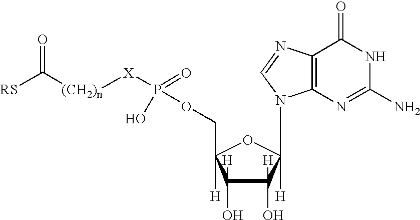
, wherein R is a hydrocarbon-containing group and X is a nucleophile.

2. The thiolester-activated nucleotide of claim 1, wherein X is sulfur.
